(19) United States
(12) Patent Application Publication
(10) Pub. No.: US 2022/0031700 A1
(43) Pub. Date: Feb. 3, 2022

(54) Treatment of Acute Respiratory Distress Syndrome (ARDS)

(71) Applicant: Melior Pharmaceuticals I, Inc., Exton, PA (US)

(72) Inventors: Andrew G. Reaume, Exton, PA (US); Gennady Smagin, Exton, PA (US)

(21) Appl. No.: 17/388,299

(22) Filed: Jul. 29, 2021

Related U.S. Application Data
(60) Provisional application No. 63/058,774, filed on Jul. 30, 2020.
(60) Provisional application No. 63/146,561, filed on Feb. 5, 2021.

(51) Int. Cl. A61K 31/513; A61P 31/16; A61K 45/06

(52) U.S. Cl. CPC A61K 31/513; A61K 45/06; A61P 31/16

(57) ABSTRACT

Methods of preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition by administering a lyn kinase activator are provided herein.

### FIELD

The present disclosure is directed, in part, to methods of treating acute respiratory distress syndrome (ARDS), pulmonary extravasation, pulmonary edema, pneumonia, fluid in the lung, plasma extravasation, and/or pulmonary conditions that develop as a result of hypercytokinemia (cytokine storm syndrome), by administering a lyn kinase activator.

### BACKGROUND

Humans infected with the SARS-CoV-2 virus have a very wide range of responses, ranging from being asymptomatic to death. In addition, those subjects who develop severe symptoms are associated with an over-reactive immune response which can sometimes persist even after viral loads are significantly reduced or even cleared (Coperchini et al., Cytokine and Growth Factor Rev., 2020, 53, 25-32). The elevated levels of several hallmark cytokines associated with this form of immune over reaction has been coined a “cytokine storm” and has been the topic of much attention in COVID-19 research. One of the effects of the “cytokine storm” is the development of pulmonary leakiness or extravasation leading to pulmonary edema and pneumonia (Coperchini et al., Cytokine and Growth Factor Rev., 2020, 53, 25-32). Certain cytokines associated with the cytokine storm are part of a signaling pathway which leads to activation of the Srk family kinases c-srk and yes which, in turn, phosphorylate membrane cadherins leading to the formation of intercellular gaps and the flow of water and plasma proteins into the lungs (pulmonary extravasation/pulmonary edema) (Mehta et al., Physiol Rev., 2006, 86, 279-367). The increase water in lung alveoli leads to reduced pulmonary gas exchange and decreased blood oxygen saturation. Ultimately sustained and pronounced reduction in oxygen saturation can lead to multi-organ failure and death. Thus, the major cause of death in COVID-19 patients is not directly related to the virus itself but rather the body's inappropriate immune response to the virus and particularly the effects of that immune response on pulmonary epithelium. Some therapeutic approaches have attempted to address the cytokine storm through various means of reducing the immune response or certain cytokines. This approach, however, can have untoward consequences as the body requires all aspects of the immune response early in the disease process in order to eliminate virus.

### SUMMARY

The present disclosure provides methods of preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition, the methods comprising administering to the mammal a compound having the formula:

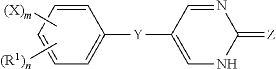

wherein: R¹ is an alkyl group; X is a halogen; Y is O, S, or NH; Z is O or S; and n is an integer from 0 to 5 and m is 0 or 1, wherein m+n is less than or equal to 5; or a pharmaceutically acceptable salt thereof.

The present disclosure also provides methods of preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition, the methods comprising administering to the mammal a compound having the formula:

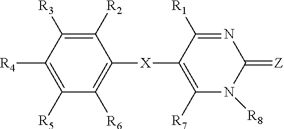

wherein: each of R₁, R₂, R₃, R₄, R₅, R₆, and R₇ is, independently, a hydrogen, alkoxy, alkyl, alkenyl, alkynyl, aryl, aryloxy, benzyl, cycloalkyl, halogen, heteroaryl, heterocycloalkyl, —CN, —OH, —NO₂, —CF₃, —CO₂H, —CO₂alkyl, or —NH₂; R₈ is an alkyl or hydrogen; X is O, S, NH, or N-akyl; and Z is O or S; or a pharmaceutically acceptable salt thereof.

The present disclosure also provides methods of preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition, the methods comprising administering to the mammal a compound having the formula:

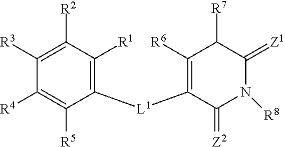

wherein:

each of R¹, R², R³, R⁴, and R⁵ is, independently, H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; or two adjacent groups of R¹, R², R³, R⁴, and R⁵ can link to form a fused cycloalkyl or fused heterocycloalkyl group, each optionally substituted by 1, 2, or 3 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; R⁶ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; R⁷ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; R⁸ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; R^{a2}, R^{b2}, R^{c2}, and R^{d2} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; or R^{c2} and R^{d2} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; Z¹ is O, S, or NR⁹; R⁹ is H, OH, C₁₋₆alkoxy, C₁₋₆haloalkyl, C₁₋₆hydroxyalkyl, aryloxy, heteroaryloxy, CN, or NO₂; Z² is O, S, or NR¹⁰; R¹⁰ is H, OH, C₁₋₆alkoxy, C₁₋₆haloalkyl, C₁₋₆hydroxyalkyl, aryloxy, heteroaryloxy, CN, or NO₂; L¹ is O, S, or NR¹¹; and R¹¹ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; or a pharmaceutically acceptable salt thereof.

The present disclosure also provides methods of preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition, the methods comprising administering to the mammal a compound having the formula:

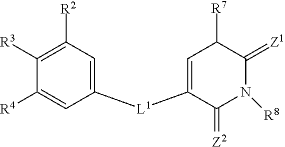

wherein: R², R³, and R⁴ are each, independently, H, halo, C₁₋₆alkyl, C₁₋₆hydroxyalkyl, or C₁₋₆haloalkyl; R⁷ is H, C₁₋₆alkyl, C(O)R^{b1}, C(O)NR^{c1}R^{d1}, or C(O)OR^{a1}; R⁸ is H, C₁₋₆alkyl, C(O)R^{b1}, C(O)NR^{c1}R^{d1}, or C(O)OR^{a1}; R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; Z¹ is O or S; Z² is O or S; and L¹ is O or S; or a pharmaceutically acceptable salt thereof.

The present disclosure also provides methods of preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition, the methods comprising administering to the mammal a compound having the formula:

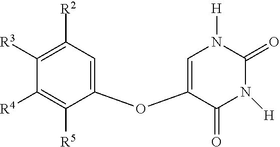

wherein: R², R³, R⁴, and R⁵ are each, independently, H, F, Cl, CH₃, SCH₃, OCH₃, C(CH₃)₃, CH(CH₃)₂, or C₂H₅; or a pharmaceutically acceptable salt thereof.

The present disclosure also provides methods of preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition, the methods comprising administering to the mammal a compound having the formula:

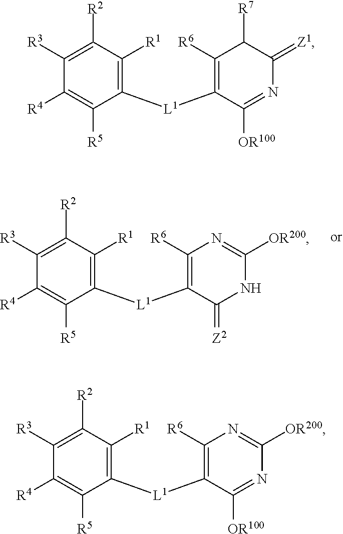

wherein:

each of R¹, R², R³, R⁴, and R⁵ is, independently, H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; or two adjacent groups of R¹, R², R³, R⁴, and R⁵ can link to form a fused cycloalkyl or fused heterocycloalkyl group, each optionally substituted by 1, 2, or 3 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; R⁶ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; R⁷ is H, C₁₋₆alkyl, C₁₋₆haloalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}, R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; or R^{c1} and R^{a1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; R^{a2}, R^{b2}, R^{c2}, and R^{d2} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; or R^{c2} and R^{d2} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; Z¹ is O, S, or NR⁹; R⁹ is H, OH, C₁₋₆alkoxy, C₁₋₆haloalkyl, C₁₋₆hydroxyalkyl, aryloxy, heteroaryloxy, CN, or NO₂; Z² is O, S, or NR¹⁰; R¹⁰ is H, OH, C₁₋₆alkoxy, C₁₋₆haloalkyl, C₁₋₆hydroxyalkyl, aryloxy, heteroaryloxy, CN, or NO₂; L¹ is O, S, or NR¹¹; R¹¹ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; R¹⁰⁰ is a hydroxyl protecting group, C₁₋₆alkyl, C₂₋₆alkenyl, C₂₋₆alkynyl, C₁₋₆haloalkyl, C₁₋₆hydroxyalkyl, C₃₋₆cycloalkyl, heterocycloalkyl, aryl, heteroaryl, C(O)R^{b1}, C(O)NR^{c1}R^{d1}, C(O)OR^{a1}, S(O)R^{b1}, S(O)NR^{c1}R^{d1}, S(O)₂R^{b1}, S(O)₂NR^{c1}R^{d1}, S(O)₂OR^{e1}, P(O)OR^{f1}OR^{g1}, or Si(R^{h1})₃, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4 or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; R²⁰⁰ is a hydroxyl protecting group, C₁₋₆alkyl, C₂₋₆alkenyl, C₂₋₆alkynyl, C₁₋₆haloalkyl, C₁₋₆hydroxyalkyl, C₃₋₆cycloalkyl, heterocycloalkyl, aryl, heteroaryl, C(O)R^{b1}, C(O)NR^{c1}R^{d1}, C(O)OR^{a1}, S(O)R^{b1}, S(O)NR^{c1}R^{d1}, S(O)₂R^{b1}, S(O)₂NR^{c1}R^{d1}, S(O)₂OR^{e1}, P(O)OR^{f1}OR^{g1}, or Si(R^{h1})₃, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4 or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; each R^{e1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, cycloalkylalkyl, heterocycloalkylalkyl, arylalkyl, or heteroarylalkyl; each R^{f1} is, independently, H, C₁₋₆alkyl, C₁₋₆haloalkyl, C₁₋₆hydroxyalkyl, C₂₋₆alkenyl, (C₁₋₆alkoxy)-C₁₋₆alkyl, C₂₋₆alkynyl, aryl, cycloalkyl, heteroaryl, heterocycloalkyl, arylalkyl, cycloalkylalkyl, heteroarylalkyl, or heterocycloalkylalkyl; each R^{g1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, or heterocycloalkyl; and each R^{h1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, cycloalkylalkyl, heterocycloalkylalkyl, arylalkyl, or heteroarylalkyl; or a pharmaceutically acceptable salt thereof.

### BRIEF DESCRIPTION OF THE DRAWINGS

FIG. 1 shows MLR-1023 exhibits a dose-dependent reduction of pulmonary extravasation by two independent measures in a mouse model of cytokine storm.

FIG. 2 shows MLR-1023 provides prophylactic benefit against pulmonary edema in a model of cytokine storm which lasts well after drug levels have cleared.

FIG. 3 shows MLR-1023 does not alter cytokine levels in a model of cytokine storm.

FIG. 4 shows daily body weights of animals treated with Influenza virus and MLR-1023.

FIG. 5 shows daily survival rate of animals treated with Influenza virus and MLR-1023.

FIG. 6 shows MLR-1023 significantly reduced wet weight/dry weight ratio and thereby reduced pulmonary edema in influenza infected mice.

FIG. 7 shows MLR-1023 did not alter any immunoglobulin level in influenza infected mice on Day 12 after infection when MLR-1023 was administed daily over the course of the infection, compared to influenza-infected/vehicle treated controls.

### DESCRIPTION OF EMBODIMENTS

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “alkoxy” means a straight or branched —O-alkyl group of 1 to 20 carbon atoms, including, but not limited to, methoxy, ethoxy, n-propoxy, isopropoxy, t-butoxy, and the like. In some embodiments, the alkoxy chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length. An alkoxy group can be unsubstituted or substituted with one or two suitable substituents.

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, nonyl, decyl, 2,2,4-trimethylpentyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like. An alkyl group can be unsubstituted or substituted with one or two suitable substituents.

As used herein, the term “alkenyl” means a straight or branched alkyl group having one or more double carbon-carbon bonds and 2-20 carbon atoms, including, but not limited to, ethenyl, 1-propenyl, 2-propenyl, 2-methyl-1-propenyl, 1-butenyl, 2-butenyl, vinyl, allyl, pentenyl, hexenyl, butadienyl, pentadienyl, hexadienyl, 2-ethylhexenyl, 2-propyl-2-butenyl, 4-(2-methyl-3-butene)-pentenyl and the like. In some embodiments, the alkenyl chain is from 2 to 10 carbon atoms in length, from 2 to 8 carbon atoms in length, from 2 to 6 carbon atoms in length, or from 2 to 4 carbon atoms in length. The double bond of an alkenyl group can be unconjugated or conjugated to another unsaturated group. An alkenyl group can be unsubstituted or substituted with one or two suitable substituents.

As used herein, the term “alkynyl” means a straight or branched alkyl group having one or more triple carbon-carbon bonds and 2-20 carbon atoms, including, but not limited to, acetylene, 1-propylene, 2-propylene, ethynyl, propynyl, butynyl, pentynyl, hexynyl, methylpropynyl, 4-methyl-1-butynyl, 4-propyl-2-pentynyl, and 4-butyl-2-hexynyl, and the like. In some embodiments, the alkynyl chain is 2 to 10 carbon atoms in length, from 2 to 8 carbon atoms in length, from 2 to 6 carbon atoms in length, or from 2 to 4 carbon atoms in length. The triple bond of an alkynyl group can be unconjugated or conjugated to another unsaturated group. An alkynyl group can be unsubstituted or substituted with one or two suitable substituents.

As used herein, the term “animal” includes, but is not limited to, humans and non-human vertebrates such as wild, domestic, and farm animals.

As used herein, the term “aryl” means a monocyclic, bicyclic, or polycyclic (e.g., having 2, 3 or 4 fused rings) aromatic hydrocarbons. In some embodiments, aryl groups have from 6 to 20 carbon atoms or from 6 to 10 carbon atoms. Examples of aryl groups include, but are not limited to, phenyl, naphthyl, anthracenyl, phenanthrenyl, indanyl, indenyl, tolyl, fluorenyl, tetrahydronaphthyl, azulenyl, naphthyl, 5,6,7,8-tetrahydronaphthyl, and the like. An aryl group can be unsubstituted or substituted with one or two suitable substituents.

As used herein, the term “aryloxy” means an —O-aryl group, wherein aryl is as defined herein. An aryloxy group can be unsubstituted or substituted with one or two suitable substituents. The aryl ring of an aryloxy group can be a monocyclic ring, wherein the ring comprises 6 carbon atoms, referred to herein as “(C₆)aryloxy.”

As used herein, the term “benzyl” means —CH₂-phenyl.

As used herein, the term “carbonyl” group is a divalent group of the formula —C(O)—.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the term, “compound” means all stereoisomers, tautomers, and isotopes of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “cycloalkyl” means non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to 15, from 3 to 10, from 3 to 8, from 3 to 6, from 4 to 6, from 3 to 5, or 5 or 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl or 1H-inden-2(3H)-one-1-yl). A cycloalkyl group can be unsubstituted or substituted by one or two suitable substituents.

As used herein, the term “halogen” means fluorine, chlorine, bromine, or iodine. Correspondingly, the meaning of the terms “halo” and “Hal” encompass fluoro, chloro, bromo, and iodo.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, 2 to 5 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, pyranyl, oxadiazolyl, isoxazolyl, triazolyl, thianthrenyl, pyrazolyl, indolizinyl, isoindolyl, isobenzofuranyl, benzoxazolyl, xanthenyl, 2H-pyrrolyl, pyrrolyl, 3H-indolyl, 4H-quinolizinyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furazanyl, phenoxazinyl, pyrazyl, phienyl, groups, and the like. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine. A heteroaryl group can be unsubstituted or substituted with one or two suitable substituents.

As used herein, the term “heterocycle” or “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring. Particularly useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Hetercycloalkyl groups can be mono or polycyclic (e.g., fused, bridged, or spiro systems). In some embodiments, the heterocycloalkyl group has from 1 to 20 carbon atoms, or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, piperidinyl, benzo-1,4-dioxane, pyrrolidinyl, isoxazolidinyl, oxazolidinyl, isothiazolidinyl, pyrazolidinyl, thiazolidinyl, imidazolidinyl, pyrrolidino, piperidino, morpholinyl, thiomorpholinyl, pyranyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, isoindolin-1-one-3-yl, 4,5,6,7-tetrahydrothieno[2,3-c]pyridine-5-yl, 5,6-dihydrothieno [2,3-c]pyridin-7(4H)-one-5-yl, and 3,4-dihydroisoquinolin-1(2H)-one-3y1 groups. Ring-forming carbon atoms and heteroatoms of the heterocycloalkyl group can be optionally substituted by oxo or sulfido. A heterocycloalkyl group can be unsubstituted or substituted with one or two suitable substituents.

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof.

As used herein, the phrase “integer from 1 to 5” means 1, 2, 3, 4, or 5.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, malic, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, malate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present invention also includes quaternary ammonium salts of the compounds described herein, where the compounds have one or more tertiary amine moiety.

As used herein, the term “phenyl” means —C₆H₅. A phenyl group can be unsubstituted or substituted with one, two, or three suitable substituents.

As used herein, the terms “prevention” or “preventing” mean a reduction of the risk of acquiring a particular disease, condition, or disorder.

As used herein, the phrase “suitable substituent” or “substituent” means a group that does not nullify the synthetic or pharmaceutical utility of the compounds described herein or the intermediates useful for preparing them. Examples of suitable substituents include, but are not limited to: C₁-C₆alkyl, C₁-C₆alkenyl, C₁-C₆alkynyl, C₅-C₆aryl, C₁-C₆alkoxy, C₃-C₅heteroaryl, C₃-C₆cycloalkyl, C₅-C₆aryloxy, —CN, —OH, oxo, halo, haloalkyl, —NO₂, —CO₂H, —NH₂, —CHO, —NH(C₁-C₈alkyl), —N(C₁-C₈alkyl)₂, —NH(C₆aryl), —N(C₅-C₆aryl)₂, —CO(C₁-C₆alkyl), —CO((C₅-C₆)aryl), —CO₂((C₁-C₆)alkyl), and —CO₂((C₅-C₆)aryl). One of skill in art can readily choose a suitable substituent based on the stability and pharmacological and synthetic activity of the compounds described herein.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects, or at least one adverse effect of a disorder is ameliorated or alleviated. The amount needed to elicit the therapeutic response can be determined based on the age, health, size and sex of the subject. Optimal amounts can also be determined based on monitoring of the subject's response to treatment.

As used herein, the terms “treat,” “treated,” or “treating” mean therapeutic treatment measures wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment may include eliciting a clinically significant response without excessive levels of side effects. Treatment may also include prolonging survival as compared to expected survival if not receiving treatment.

The compounds of the disclosure are identified herein by their chemical structure and/or chemical name. Where a compound is referred to by both a chemical structure and a chemical name, and that chemical structure and chemical name conflict, the chemical structure is determinative of the compound's identity.

At various places in the present specification, substituents of compounds may be disclosed in groups or in ranges. It is specifically intended that the invention include each and every individual subcombination of the members of such groups and ranges. For example, the term “C₁₋₆alkyl” is specifically intended to individually disclose methyl, ethyl, propyl, C₄alkyl, C₅alkyl, and C₆alkyl, linear and/or branched.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form, for example,

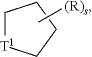

then it is understood that substituent R can occur “s” number of times on the ring, and R can be a different moiety at each occurrence. Further, in the above example, where the variable T¹ is defined to include hydrogens, such as when T¹ is CH₂, NH, etc., any H can be replaced with a substituent.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable sub-combination.

It is understood that the present disclosure encompasses the use, where applicable, of stereoisomers, diastereomers and optical stereoisomers of the compounds of the disclosure, as well as mixtures thereof. Additionally, it is understood that stereoisomers, diastereomers, and optical stereoisomers of the compounds of the disclosure, and mixtures thereof, are within the scope of the disclosure. By way of non-limiting example, the mixture may be a racemate or the mixture may comprise unequal proportions of one particular stereoisomer over the other. Additionally, the compounds can be provided as a substantially pure stereoisomers, diastereomers and optical stereoisomers (such as epimers).

The compounds described herein may be asymmetric (e.g., having one or more stereocenters). All stereoisomers, such as enantiomers and diastereomers, are intended to be included within the scope of the disclosure unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds are also included within the scope of the disclosure and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, fractional recrystallizaion using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds also include hydrates and solvates, as well as anhydrous and non-solvated forms.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or pharmaceutically acceptable salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or pharmaceutically acceptable salt thereof. Methods for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti arrangement of the two carbonyl moiety and alter the periodicity of the compound. Pyromellitic anhydride represents still another alternative to simple amide linkages which can alter the conformation and physical properties of the compound. Modern methods of solid phase organic chemistry now allow the synthesis of homodisperse compounds with molecular weights approaching 5,000 Daltons. Other substitution patterns are equally effective.

The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds described herein.

Compounds containing an amine function can also form N-oxides. A reference herein to a compound that contains an amine function also includes the N-oxide. Where a compound contains several amine functions, one or more than one nitrogen atom can be oxidized to form an N-oxide. Examples of N-oxides include N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g., a peroxycarboxylic acid).

The present disclosure provides methods of preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition, the methods comprising administering to the mammal any one or more of the lyn kinase activators described herein, or compositions comprising the same.

In some embodiments, the methods prevent the development of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the methods prevent the development of pulmonary extravasation in a mammal having a medical condition. In some embodiments, the methods prevent the development of pulmonary edema in a mammal having a medical condition. In some embodiments, the methods prevent the development of pneumonia in a mammal having a medical condition. In some embodiments, the methods prevent the development of fluid in the lung in a mammal having a medical condition. In some embodiments, the methods prevent the development of acute respiratory distress syndrome in a mammal having a medical condition. In some embodiments, the methods prevent the development of plasma extravasation in a mammal having a medical condition.

In some embodiments, the methods delay the development of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the methods delay the development of pulmonary extravasation in a mammal having a medical condition. In some embodiments, the methods delay the development of pulmonary edema in a mammal having a medical condition. In some embodiments, the methods delay the development of pneumonia in a mammal having a medical condition. In some embodiments, the methods delay the development of fluid in the lung in a mammal having a medical condition. In some embodiments, the methods delay the development of acute respiratory distress syndrome in a mammal having a medical condition. In some embodiments, the methods delay the development of plasma extravasation in a mammal having a medical condition.

In some embodiments, the methods reduce the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the methods reduce the severity of pulmonary extravasation in a mammal having a medical condition. In some embodiments, the methods reduce the severity of pulmonary edema in a mammal having a medical condition. In some embodiments, the methods reduce the severity of pneumonia in a mammal having a medical condition. In some embodiments, the methods reduce the severity of fluid in the lung in a mammal having a medical condition. In some embodiments, the methods reduce the severity of acute respiratory distress syndrome in a mammal having a medical condition. In some embodiments, the methods reduce the severity of plasma extravasation in a mammal having a medical condition.

In some embodiments, the medical condition is a viral infection. In some embodiments, the viral infection is an influenza virus infection, a corona virus infection, a human rhinovirus (HRV) infection, a respiratory syncytial virus (RSV) infection, a parainfluenza virus (PIV) infection, a human metapneumovirus (hMPV) infection, or an adenovirus infection. In some embodiments, the viral infection is an influenza virus infection. In some embodiments, the viral infection is a corona virus infection. In some embodiments, the viral infection is an HRV infection. In some embodiments, the viral infection is aa RSV infection. In some embodiments, the viral infection is a PIV infection. In some embodiments, the viral infection is an hMPV infection. In some embodiments, the viral infection is an adenovirus infection. In some embodiments, the influenza virus infection is an influenza A H1N1 virus infection. In some embodiments, the corona virus infection is a severe acute respiratory syndrome coronavirus 2 (SARS-CoV-2) infection, a severe acute respiratory syndrome coronavirus 1 (SARS-CoV-1) infection, or a Middle East respiratory syndrome virus (MERS) infection. In some embodiments, the corona virus infection is a SARS-CoV-2 infection. In some embodiments, the corona virus infection is a SARS-CoV-1 infection. In some embodiments, the corona virus infection is a MERS infection.

In some embodiments, the medical condition is a bacterial infection. In some embodiments, the bacterial infection is bacterial pneumonia or sepsis. In some embodiments, the bacterial infection is bacterial pneumonia. In some embodiments, the bacterial infection is sepsis.

In some embodiments, the medical condition is a cardiovascular condition. In some embodiments, the cardiovascular condition is acute heart failure.

In some embodiments, the medical condition is inhalation of a harmful agent. In some embodiments, the harmful agent is smoke or a noxious chemical fume. In some embodiments, the harmful agent is smoke. In some embodiments, the harmful agent is a noxious chemical fume.

In some embodiments, the medical condition is a head or chest injury.

In some embodiments, the lyn kinase activator is of the formula:

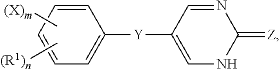

wherein: R¹ is an alkyl group; X is a halogen; Y is O, S, or NH; Z is O or S; and n is an integer from 0 to 5 and m is 0 or 1, wherein m+n is less than or equal to 5; or a pharmaceutically acceptable salt thereof. In some embodiments, the alkyl group is methyl and n is 1. In some embodiments, the halogen is chlorine and m is 1. In some embodiments, Y is O. In some embodiments, Z is O. In some embodiments, R¹ is methyl, Y is O, Z is O, n is 1, and m is 0. In some embodiments, R¹ is in the meta position. In some embodiments, X is chlorine, Y is O, Z is O, n is 0, and m is 1. In some embodiments, X is in the meta position.

In some embodiments, the lyn kinase activator is of the formula:

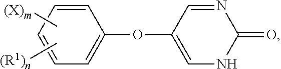

wherein: R¹ is an alkyl group; X is a halogen; and n is an integer from 0 to 5 and m is 0 or 1, wherein m+n is less than or equal to 5; or a pharmaceutically acceptable salt thereof. In some embodiments, the alkyl group is methyl and n is 1. In some embodiments, the halogen is chlorine and m is 1. In some embodiments, R¹ is methyl, n is 1, and m is 0. In some embodiments, R¹ is in the meta position. In some embodiments, X is chlorine, n is 0, and m is 1. In some embodiments, X is in the meta position.

In some embodiments, the lyn kinase activator is of the formula:

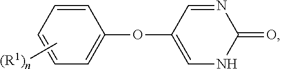

wherein R¹ is an alkyl group and n is an integer from 0 to 5; or a pharmaceutically acceptable salt thereof. In some embodiments, R¹ is methyl, n is 1. In some embodiments, R¹ is in the meta position.

In some embodiments, the lyn kinase activator is of the formula:

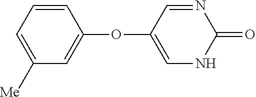 [SMILES: Cc1cccc(Oc2cnc(=O)[nH]c2)c1]

(Compound 102; MLR-1023; tolimidone), or a pharmaceutically acceptable salt thereof.

In some embodiments, the lyn kinase activator is of the formula:

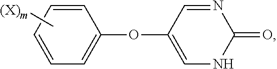

wherein X is a halogen and m is an integer from 0 to 1; or a pharmaceutically acceptable salt thereof. In some embodiments, X is chloro and m is 1. In some embodiments, X is in the meta position.

In some embodiments, the lyn kinase activator is of the formula:

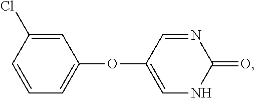 [SMILES: O=c1ncc(Oc2cccc(Cl)c2)c[nH]1]

or a pharmaceutically acceptable salt thereof.

In some embodiments, the lyn kinase activator is of the formula:

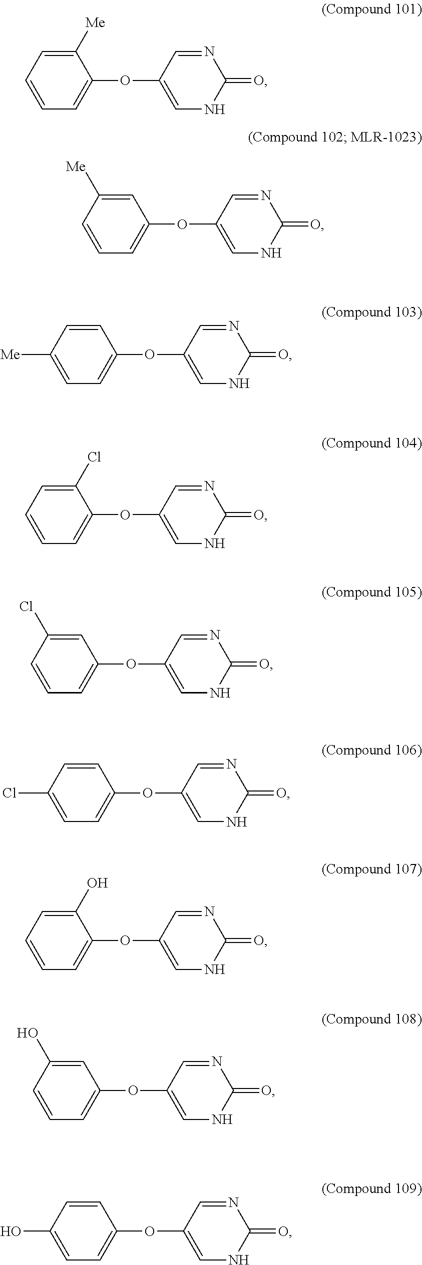

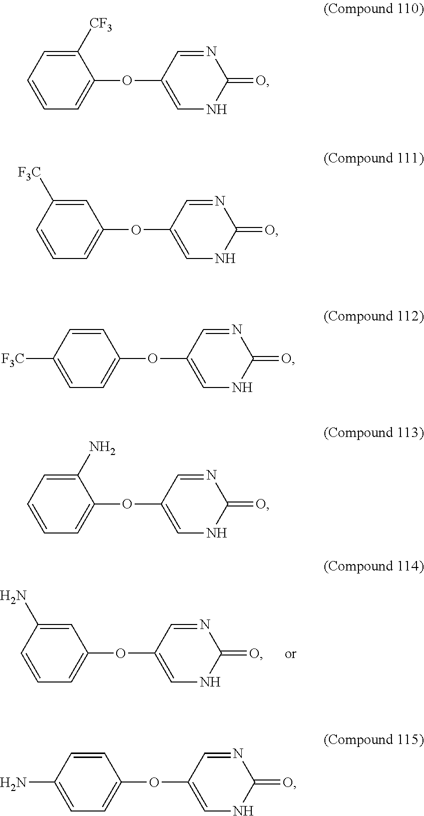 [SMILES: O=c1ncc(Oc2ccccc2C(F)(F)F)c[nH]1 ; O=c1ncc(Oc2cccc(C(F)(F)F)c2)c[nH]1 ; O=c1ncc(Oc2ccc(C(F)(F)F)cc2)c[nH]1 ; Nc1ccccc1Oc1cnc(=O)[nH]c1 ; Nc1cccc(Oc2cnc(=O)[nH]c2)c1 ; Nc1ccc(Oc2cnc(=O)[nH]c2)cc1]

or a pharmaceutically acceptable salt thereof.

In some embodiments, the lyn kinase activator is of the formula:

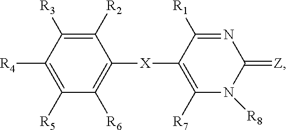

wherein: each of R₁, R₂, R₃, R₄, R₅, R₆, and R₇ is, independently, a hydrogen, alkoxy, alkyl, alkenyl, alkynyl, aryl, aryloxy, benzyl, cycloalkyl, halogen, heteroaryl, heterocycloalkyl, —CN, —OH, —NO₂, —CF₃, —CO₂H, —CO₂alkyl, or —NH₂; R₈ is an alkyl or hydrogen; X is O, S, NH, or N-akyl; and Z is O or S; or a pharmaceutically acceptable salt thereof. In some embodiments, R₈ is alkyl. In some embodiments, R₈ is methyl. In some embodiments, R₈ is hydrogen. In some embodiments, X is oxygen. In some embodiments, Z is oxygen. In some embodiments, at least one of R₂-R₆ is alkyl. In some embodiments, at least one of R₂-R₆ is methyl. In some embodiments, at least one of R₂-R₆ is halogen. In some embodiments, at least one of R₂-R₆ is chloro. In some embodiments, at least one of R₂-R₆ is —CN, —OH, —NO₂, —CF₃, —CO₂H, —NH₂, or alkoxy. In some embodiments, R₂ is alkyl, each of R₁ and R₃-R₈ is hydrogen, and X and Z are O. In some embodiments, R₂ is methyl. In some embodiments, R₂ is a halogen, each of R₁ and R₃-R₈ is hydrogen, and X and Z are O. In some embodiments, R₂ is chloro. In some embodiments, R₃ is alkyl, each of R₁, R₂ and R₄-R₈ is hydrogen, and X and Z are O. In some embodiments, R₃ is methyl. In some embodiments, R₃ is a halogen, each of R₁, R₂, and R₄-R₈ is hydrogen, and X and Z are O. In some embodiments, R₃ is chloro. In some embodiments, R₄ is alkyl, each of R₁-R₃ and R₅-R₈ is hydrogen, and X and Z are O. In some embodiments, R₄ is methyl. In some embodiments, R₄ is a halogen, each of R₁-R₃ and R₅-R₈ is hydrogen, and X and Z are O. In some embodiments, R₄ is chloro. In some embodiments, R₅ is —CF₃, each of R₁-R₄ and R₆-R₈ is hydrogen, and X and Z are O. In some embodiments, R₅ is —NH₂, each of R₁-R₄ and R₆-R₈ is hydrogen, and X and Z are O. In some embodiments, R₆ is —CF₃, each of R₁-R₅ and R₇-R₈ is hydrogen, and X and Z are O. In some embodiments, R₆ is —NH₂, each of R₁-R₅ and R₇-R₈ is hydrogen, and X and Z are O.

In some embodiments, the lyn kinase activator is of the formula:

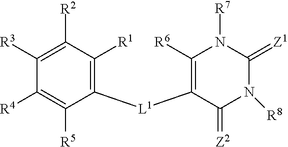

wherein:

R¹ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R² is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R³ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}.

R⁴ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S (O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁵ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

or two adjacent groups of R¹, R², R³, R⁴, and R⁵ can link to form a fused cycloalkyl or fused heterocycloalkyl group, each optionally substituted by 1, 2, or 3 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁶ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁷ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1};

R⁸ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1};

R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

R^{a2}, R^{b2}, R^{c2}, and R^{d2} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

or R^{c2} and R^{d2} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

Z¹ is O, S, or NR⁹;

R⁹ is H, OH, C₁₋₆alkoxy, C₁₋₆haloalkyl, C₁₋₆hydroxyalkyl, aryloxy, heteroaryloxy, CN, or NO₂;

Z² is O, S, or NR¹⁰;

R¹⁰ is H, OH, C₁₋₆alkoxy, C₁₋₆haloalkyl, C₁₋₆hydroxyalkyl, aryloxy, heteroaryloxy, CN, or NO₂;

L¹ is O, S, or NR¹¹; and

R¹¹ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; or a pharmaceutically acceptable salt thereof.

In some embodiments, the lyn kinase activator is of the formula:

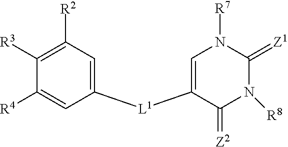

wherein:

R², R³, and R⁴ are each, independently, H, halo, C₁₋₆alkyl, C₁₋₆hydroxyalkyl, or C₁₋₆haloalkyl;

R⁷ is H, C₁₋₆alkyl, C(O)R^{b1}, C(O)NR^{c1}R^{d1}, or C(O)OR^{a1};

R⁸ is H, C₁₋₆alkyl, C(O)R^{b1}, C(O)NR^{c1}R^{d1}, or C(O)OR^{a1};

R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

Z¹ is O or S;

Z² is O or S; and

L¹ is O or S; or a pharmaceutically acceptable salt thereof.

In some embodiments, the lyn kinase activator is of the formula:

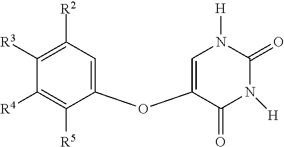

wherein: R², R³, R⁴, and R⁵ are each, independently, H, F, Cl, CH₃, SCH₃, OCH₃, C(CH₃)₃, CH(CH₃)₂, or C₂H₅; or a pharmaceutically acceptable salt thereof.

In some embodiments, the lyn kinase activator is of the formula:

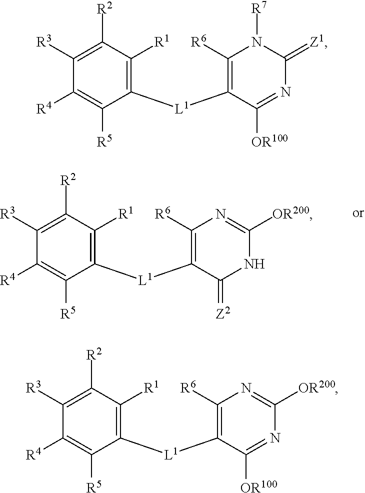

wherein:

R¹ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R² is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R³ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1},NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁴ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁵ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

or two adjacent groups of R¹, R², R³, R⁴, and R⁵ can link to form a fused cycloalkyl or fused heterocycloalkyl group, each optionally substituted by 1, 2, or 3 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁶ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2},

R⁷ is H, C₁₋₆alkyl, C₁₋₆haloalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1};

R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

R^{a2}, R^{b2}, R^{c2}, and R^{d2} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

or R^{c2} and R^{d2} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

Z¹ is O, S, or NR⁹;

R⁹ is H, OH, C₁₋₆alkoxy, C₁₋₆haloalkyl, C₁₋₆hydroxyalkyl, aryloxy, heteroaryloxy, CN, or NO₂;

Z² is O, S, or NR¹⁰;

R¹⁰ is H, OH, C₁₋₆alkoxy, C₁₋₆haloalkyl, C₁₋₆hydroxyalkyl, aryloxy, heteroaryloxy, CN, or NO₂;

L¹ is O, S, or NR¹¹;

R¹¹ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1};

R¹⁰⁰ is a hydroxyl protecting group, C₁₋₆alkyl, C₂₋₆alkenyl, C₂₋₆alkynyl, C₁₋₆haloalkyl, C₁₋₆hydroxyalkyl, C₃₋₆cycloalkyl, heterocycloalkyl, aryl, heteroaryl, C(O)R^{b1}, C(O)NR^{c1}R^{d1}, C(O)OR^{a1}, S(O)R^{b1}, S(O)NR^{c1}R^{d1}, S(O)₂R^{b1}, S(O)₂NR^{c1}R^{d1}, S(O)₂OR^{e1}, P(O)OR^{f1}OR^{g1}, or Si(R^{h1})₃, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4 or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R²⁰⁰ is a hydroxyl protecting group, C₁₋₆alkyl, C₂₋₆alkenyl, C₂₋₆alkynyl, C₁₋₆haloalkyl, C₁₋₆hydroxyalkyl, C₃₋₆cycloalkyl, heterocycloalkyl, aryl, heteroaryl, C(O)R^{b1}, C(O)NR^{c1}R^{d1}, C(O)OR^{a1}, S(O)R^{b1}, S(O)NR^{c1}R^{d1}, S(O)₂R^{b1}, S(O)₂NR^{c1}R^{d1}, S(O)₂OR^{e1}, P(O)OR^{f1}OR^{g1}, or Si(R^{h1})₃, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4 or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

each R^{e1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, cycloalkylalkyl, arylalkyl, heterocycloalkylalkyl, or heteroarylalkyl;

each R^{f1} is, independently, H, C₁₋₆alkyl, C₁₋₆haloalkyl, C₁₋₆hydroxyalkyl, C₂₋₆alkenyl, (C₁₋₆alkoxy)-C₁₋₆alkyl, C₂₋₆alkynyl, aryl, cycloalkyl, heteroaryl, heterocycloalkyl, arylalkyl, cycloalkylalkyl, heteroarylalkyl, or heterocycloalkylalkyl;

each R^{g1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, or heterocycloalkyl; and

each R^{h1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, cycloalkylalkyl, arylalkyl, heterocycloalkylalkyl, or heteroarylalkyl; or a pharmaceutically acceptable salt thereof.

In some embodiments, the lyn kinase activator is a compound of the formula:

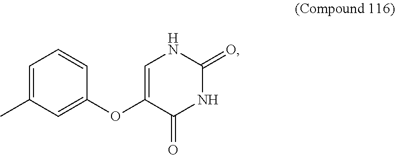 [SMILES: Cc1cccc(Oc2c[nH]c(=O)[nH]c2=O)c1]

which is also known as 5-(m-tolyloxy)pyrimidine-2,4(1H,3H)-dione.

It will be understood that the compounds are illustrative only and not intended to limit the scope of the claims to only those compounds.

The compounds described herein can be synthesized by standard organic chemistry techniques known to those of ordinary skill in the art, for example as described in U.S. Pat. Nos. 3,922,345 and 4,080,454. Preparation of the compounds described herein can involve the protection and deprotection of various chemical groups. The need for protection and deprotection, and the selection of appropriate protecting groups, can be readily determined by one skilled in the art. Suitable hydroxyl protecting groups include, but are not limited to, tert-butyldimethylsilyl (TBS), methoxymethyl ether (MOM), tetrahydropyranyl ether (THP), t-Butyl ether, allyl ether, benzyl ether, t-Butyldimethylsilyl ether (TBDMS), t-Butyldiphenylsilyl ether (TBDPS), acetic acid ester, and the like.

In some embodiments, the compositions described herein are pharmaceutical compositions and comprise a pharmaceutically acceptable carrier, vehicle, diluent, or excipient.

Vehicles include, but are not limited to a diluent, adjuvant, excipient, or carrier with which a compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds and pharmaceutically acceptable vehicles are preferably sterile. Water is a suitable vehicle when the compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule. Other examples of suitable pharmaceutical vehicles are described in Remington's Pharmaceutical Sciences, A. R. Gennaro (Editor) Mack Publishing Co.

The compounds can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH 7.0), 0.9% saline, and 1.2% saline, and any combination thereof. In some embodiments, excipient is chosen from propylene glycol, purified water, and glycerin.

In some embodiments, the formulation can be lyophilized to a solid and reconstituted with, for example, water prior to use.

When administered to a human, the compounds can be sterile. Water is a suitable carrier when the compound is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition can be in the form of a liquid wherein the active agent (i.e., one of the facially amphiphilic polymers or oligomers disclosed herein) is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

In some embodiments, one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

One or more antioxidants can also be included in the compositions. Suitable antioxidants include, but are not limited to, ascorbic acid, sodium metabisulfite, sodium bisulfite, acetylcysteine, polyquaternium-1, benzalkonium chloride, thimerosal, chlorobutanol, methyl paraben, propyl paraben, phenylethyl alcohol, edetate disodium, sorbic acid, or other agents know to those of skill in the art. Such preservatives are typically employed at a level of from about 0.001% to about 1.0% by weight.

In some embodiments, the compounds are solubilized at least in part by an acceptable solubilizing agent. Certain acceptable nonionic surfactants, for example polysorbate 80, can be useful as solubilizing agents, as can acceptable glycols, polyglycols, e.g., polyethylene glycol 400 (PEG-400), and glycol ethers. Suitable solubilizing agents for solution and solution/suspension compositions are cyclodextrins. Suitable cyclodextrins include α-cyclodextrin, β-cyclodextrin, γ-cyclodextrin, alkylcyclodextrins (such as, methyl-β-cyclodextrin, dimethyl-β-cyclodextrin, diethyl-β-cyclodextrin), hydroxyalkylcyclodextrins (such as, hydroxyethyl-β-cyclodextrin, hydroxypropyl-β-cyclodextrin), carboxyalkylcyclodextrins (such as, carboxymethyl-β-cyclodextrin), sulfoalkylether cyclodextrins (such as, sulfobutylether-β-cyclodextrin), and the like. An acceptable cyclodextrin can optionally be present in a composition at a concentration from about 1 to about 200 mg/ml, from about 5 to about 100 mg/ml, or from about 10 to about 50 mg/ml.

In some embodiments, the composition contains a suspending agent. For example, in those embodiments in which the composition is an aqueous suspension or solution/suspension, the composition can contain one or more polymers as suspending agents. Useful polymers include, but are not limited to, water-soluble polymers such as cellulosic polymers, for example, hydroxypropyl methylcellulose, and water-insoluble polymers such as cross-linked carboxyl-containing polymers.

One or more acceptable pH adjusting agents and/or buffering agents can be included in the compositions, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, one or more acceptable surfactants, such as nonionic surfactants, or co-solvents can be included in the compositions to enhance solubility of the components of the compositions or to impart physical stability, or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbate 20, 60 and 80; polyoxyethylene/polyoxypropylene surfactants (e.g., Pluronic® F-68, F84 and P-103); cyclodextrin; or other agents known to those of skill in the art. Typically, such co-solvents or surfactants are employed in the compositions at a level of from about 0.01% to about 2% by weight.

The compounds described herein can be formulated for parenteral administration by injection, such as by bolus injection or continuous infusion. The compounds can be administered by continuous infusion subcutaneously over a period of about 15 minutes to about 24 hours. Formulations for injection can be presented in unit dosage form, such as in ampoules or in multi-dose containers, with an added preservative. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

In some embodiments, the compounds are formulated in accordance with routine procedures as a pharmaceutical composition adapted for intravenous administration to human beings. Typically, compounds for intravenous administration are solutions in sterile isotonic aqueous buffer. Where necessary, the compositions may also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The compounds described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In some embodiments, the compositions can be administered orally. Compositions for oral delivery may be in the form of tablets, lozenges, aqueous or oily suspensions, granules, powders, emulsions, capsules, syrups, or elixirs, for example. Orally administered compositions may contain one or more additional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles can be pharmaceutical grade.

For oral administration, the compounds described herein can be formulated by combining the compounds with pharmaceutically acceptable carriers. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

In some embodiments, the compounds can be delivered in a controlled release system. In some embodiments, a pump may be used. In some embodiments, polymeric materials can be used. In some embodiments, a controlled-release system can be placed in proximity of the target of the compounds described herein, such as the lung, thus requiring only a fraction of the systemic dose. In some embodiments, the compounds described herein can be delivered in a vesicle, in particular a liposome.

For administration by inhalation, the compounds described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

In transdermal administration, the compounds can be applied to a plaster, or can be applied by transdermal, therapeutic systems that are consequently supplied to the organism. In some embodiments, the compounds are present in creams, solutions, powders, fluid emulsions, fluid suspensions, semi-solids, ointments, pastes, gels, jellies, and foams, or in patches containing any of the same.

The amount of a lyn kinase activator that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally from about 0.001 mg to about 200 mg of a compound per kg body weight. In some embodiments, the oral dose is from about 0.01 mg to about 70 mg per kg body weight, from about 0.1 mg to about 50 mg per kg body weight, from about 0.5 mg to about 20 mg per kg body weight, from about 1 mg to about 10 mg per kg body weight, or about 5 mg of a compound per kg body weight. The dosage amounts described herein refer to total amounts administered; that is, if more than one compound is administered, the dosages correspond to the total amount of the compounds administered. Oral compositions can contain 10% to 95% active ingredient by weight. Suitable dosage ranges for oral administration are generally from about 50 μg to about 1,000 mg, from about 100 μg to about 500 mg, from about 250 μg to about 100 mg, from about 500 μg to about 50 mg, from about 1 mg to about 40 mg, from about 5 mg to about 25 mg, or from about 10 mg to about 20 mg.

Suitable dosage ranges for intravenous (i.v.) administration are from about 0.01 mg to about 100 mg per kg body weight, from about 0.1 mg to about 35 mg per kg body weight, and from about 1 mg to about 10 mg per kg body weight. Suitable dosage ranges for i.v. administration are generally from about 50 μg to about 1,000 mg, from about 100 μg to about 500 mg, from about 250 μg to about 100 mg, from about 500 μg to about 50 mg, from about 1 mg to about 40 mg, from about 5 mg to about 25 mg, or from about 10 mg to about 20 mg. Suitable dosage ranges for intranasal administration are generally from about 0.01 pg/kg body weight to about 1 mg/kg body weight. Recommended dosages for intradermal, intramuscular, intraperitoneal, subcutaneous, epidural, sublingual, intracerebral, intravaginal, transdermal administration or administration by inhalation are in the range of from about 0.001 mg to about 200 mg per kg of body weight. Suitable doses of the compounds for topical administration are in the range of about 0.001 mg to about 1 mg, depending on the area to which the compound is administered. Effective doses may be extrapolated from dose-response curves derived from in vitro or animal model test systems.

In some embodiments, the compound is administered after the mammal has been diagnosed as having the medical condition but before the development of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome). In some embodiments, the compound is administered to the mammal every 1 to 3 hours, every 4 to 6 hours, every 7 to 9 hours, every 10 to 12 hours, every 13 to 15 hours, every 16 to 18 hours, every 19 to 21 hours, or every 22 to 24 hours after the mammal has been diagnosed as having the medical condition. In some embodiments, the compound is administered to the mammal every 1 to 3 hours after the mammal has been diagnosed as having the medical condition. In some embodiments, the compound is administered to the mammal every 4 to 6 hours after the mammal has been diagnosed as having the medical condition. In some embodiments, the compound is administered to the mammal every 7 to 9 hours after the mammal has been diagnosed as having the medical condition. In some embodiments, the compound is administered to the mammal every 10 to 12 hours after the mammal has been diagnosed as having the medical condition. In some embodiments, the compound is administered to the mammal every 13 to 15 hours after the mammal has been diagnosed as having the medical condition. In some embodiments, the compound is administered to the mammal every 16 to 18 hours after the mammal has been diagnosed as having the medical condition. In some embodiments, the compound is administered to the mammal every 19 to 21 hours after the mammal has been diagnosed as having the medical condition. In some embodiments, the compound is administered to the mammal every 22 to 24 hours after the mammal has been diagnosed as having the medical condition.

In some embodiments, the amount of the compound administered to the mammal is from about 0.01 mg/kg to about 1000 mg/kg, from about 0.01 mg/kg to about 500 mg/kg, from about 0.1 mg/kg to about 500 mg/kg, from about 1 mg/kg to about 250 mg/kg, or from about 1 mg/kg to about 150 mg/kg. In some embodiments, the amount of the compound administered to the mammal is from about 0.01 mg/kg to about 1000 mg/kg. In some embodiments, the amount of the compound administered to the mammal is from about 0.01 mg/kg to about 500 mg/kg. In some embodiments, the amount of the compound administered to the mammal is from about 0.1 mg/kg to about 500 mg/kg. In some embodiments, the amount of the compound administered to the mammal is from about 1 mg/kg to about 250 mg/kg. In some embodiments, the amount of the compound administered to the mammal is from about 1 mg/kg to about 150 mg/kg.

In some embodiments, the amount of the compound administered to the mammal is from about 50 μg to about 1,000 mg, from about 100 μg to about 500 mg, from about 250 μg to about 100 mg, from about 500 μg to about 50 mg, from about 1 mg to about 40 mg, from about 5 mg to about 25 mg, or from about 10 mg to about 20 mg. In some embodiments, the amount of the compound administered to the mammal is from about 50 μg to about 1,000 mg. In some embodiments, the amount of the compound administered to the mammal is from about 100 μg to about 500 mg. In some embodiments, the amount of the compound administered to the mammal is from about 250 μg to about 100 mg. In some embodiments, the amount of the compound administered to the mammal is from about 500 μg to about 50 mg. In some embodiments, the amount of the compound administered to the mammal is from about 1 mg to about 40 mg. In some embodiments, the amount of the compound administered to the mammal is from about 5 mg to about 25 mg. In some embodiments, the amount of the compound administered to the mammal is from about 10 mg to about 20 mg.

The present disclosure also provides pharmaceutical packs or kits comprising one or more containers filled with one or more compositions. In some embodiments, the container(s) can further contain a notice in the form prescribed by a governmental agency regulating the manufacture, use or sale of pharmaceuticals or biological products, which notice reflects approval by the agency of manufacture, use or sale for human administration. In some embodiments, the kit contains more than one lyn kinase activator.

In some embodiments, the compositions can be used in combination therapy with at least one other therapeutic agent. The compound and the additional therapeutic agent can act additively or synergistically. In some embodiments, a composition described herein is administered concurrently with the administration of another therapeutic agent, which can be part of the same composition as the compound or a different composition. In some embodiments, a composition described herein is administered prior or subsequent to administration of another therapeutic agent. As many of the disorders for which the compositions are useful in treating are chronic disorders, in some embodiments, the combination therapy involves alternating between administering a composition described herein and a composition comprising another therapeutic agent, e.g., to minimize the toxicity associated with a particular drug. The duration of administration of each drug or therapeutic agent can be, e.g., one month, three months, six months, or a year. In some embodiments, when a composition described herein is administered concurrently with another therapeutic agent that potentially produces adverse side effects including but not limited to toxicity, the therapeutic agent can advantageously be administered at a dose that falls below the threshold at which the adverse side is elicited.

The present compositions can also comprise, or be administered together or separately, with an additional therapeutic agent used to treat pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome). Examples of additional therapeutic agents suitable for use in treatment of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome), that can be combined with one or more of the compounds described herein include, but are not limited to, an antiviral agent such as, for example, lopinavir/ritonavir, chloroquine, hydroxylchloroquine, remdesivir, ribavirin, azithromycin, falapirivir, ivermectin, enfuvirtide, amantadine, rimantadine, pleconaril, aciclovir, zidovudine, lamivudine, formivirsen, rifampicin, zanamivir, oseltamivir, peramivir, NP-120 (ifenprodil), favilavir/favipiravir, TMJ2 (TJ003234), TZLS-501, APN01, tocilizumab, galidesivir, sarilumab, SNG001, AmnioBoost, AT-100, leronlimab, BPI-002, OYA1, artemisinin, OT-101, Sepsivac, Prezcobix (darunavir and cobicistat), baricitinib, BXT-25, or duvelisib, or any combination thereof. Antiviral agents also include vaccines and/or vaccine adjuvants such as, for example, INO-4800, mRNA-1273, BPI-002, VLP (Virus-Like Particle), modified avian vaccine, TNX-1800, recombinant subunit vaccine, ChAdOx1 nCoV-19 vaccine, AdCOVID, and BNT162, or any combination thereof. Other examples of therapeutic agents suitable for use in any of the methods described herein that can be combined with one or more of the compounds described herein include, but are not limited to, anti-inflammatory agents, such as dexamethasone, tocilizumab, sarilumab, apilimod, and other agents known to reduce inflammatory cytokine levels, or any combination thereof.

The present compositions can be administered orally. The compositions can also be administered by any other convenient route, for example, by infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer the compositions. In some embodiments, more than one composition is administered to a patient. Methods of administration include, but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The desired mode of administration is left to the discretion of the practitioner, and will depend in-part upon the site of the medical condition.

In some embodiments, it may be desirable to administer one or more compositions locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In some embodiments, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In some embodiments, the compositions can be formulated as a suppository, with traditional binders and vehicles such as triglycerides.

The present disclosure also provides compositions described herein for use in preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the compositions prevent the development of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the compositions prevent the development of pulmonary extravasation in a mammal having a medical condition. In some embodiments, the compositions prevent the development of pulmonary edema in a mammal having a medical condition. In some embodiments, the compositions prevent the development of pneumonia in a mammal having a medical condition. In some embodiments, the compositions prevent the development of fluid in the lung in a mammal having a medical condition. In some embodiments, the compositions prevent the development of acute respiratory distress syndrome in a mammal having a medical condition. In some embodiments, the compositions prevent the development of plasma extravasation in a mammal having a medical condition. In some embodiments, the compositions delay the development of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the compositions delay the development of pulmonary extravasation in a mammal having a medical condition. In some embodiments, the compositions delay the development of pulmonary edema in a mammal having a medical condition. In some embodiments, the compositions delay the development of pneumonia in a mammal having a medical condition. In some embodiments, the compositions delay the development of fluid in the lung in a mammal having a medical condition. In some embodiments, the compositions delay the development of acute respiratory distress syndrome in a mammal having a medical condition. In some embodiments, the compositions delay the development of plasma extravasation in a mammal having a medical condition. In some embodiments, the compositions reduce the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the compositions reduce the severity of pulmonary extravasation in a mammal having a medical condition. In some embodiments, the compositions reduce the severity of pulmonary edema in a mammal having a medical condition. In some embodiments, the compositions reduce the severity of pneumonia in a mammal having a medical condition. In some embodiments, the compositions reduce the severity of fluid in the lung in a mammal having a medical condition. In some embodiments, the compositions reduce the severity of acute respiratory distress syndrome in a mammal having a medical condition. In some embodiments, the compositions reduce the severity of plasma extravasation in a mammal having a medical condition.

The present disclosure also provides any one or more of the lyn kinase activators described herein for use in preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the lyn kinase activators prevent the development of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the lyn kinase activators prevent the development of pulmonary extravasation in a mammal having a medical condition. In some embodiments, the lyn kinase activators prevent the development of pulmonary edema in a mammal having a medical condition. In some embodiments, the lyn kinase activators prevent the development of pneumonia in a mammal having a medical condition. In some embodiments, the lyn kinase activators prevent the development of fluid in the lung in a mammal having a medical condition. In some embodiments, the lyn kinase activators prevent the development of acute respiratory distress syndrome in a mammal having a medical condition. In some embodiments, the lyn kinase activators prevent the development of plasma extravasation in a mammal having a medical condition. In some embodiments, the lyn kinase activators delay the development of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the lyn kinase activators delay the development of pulmonary extravasation in a mammal having a medical condition. In some embodiments, the lyn kinase activators delay the development of pulmonary edema in a mammal having a medical condition. In some embodiments, the lyn kinase activators delay the development of pneumonia in a mammal having a medical condition. In some embodiments, the lyn kinase activators delay the development of fluid in the lung in a mammal having a medical condition. In some embodiments, the lyn kinase activators delay the development of acute respiratory distress syndrome in a mammal having a medical condition. In some embodiments, the lyn kinase activators delay the development of plasma extravasation in a mammal having a medical condition. In some embodiments, the lyn kinase activators reduce the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition. In some embodiments, the lyn kinase activators reduce the severity of pulmonary extravasation in a mammal having a medical condition. In some embodiments, the lyn kinase activators reduce the severity of pulmonary edema in a mammal having a medical condition. In some embodiments, the lyn kinase activators reduce the severity of pneumonia in a mammal having a medical condition. In some embodiments, the lyn kinase activators reduce the severity of fluid in the lung in a mammal having a medical condition. In some embodiments, the lyn kinase activators reduce the severity of acute respiratory distress syndrome in a mammal having a medical condition. In some embodiments, the lyn kinase activators reduce the severity of plasma extravasation in a mammal having a medical condition.

The present disclosure also provides compositions described herein for use in preparation of a medicament for preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition.

The present disclosure also provides any one or more of the lyn kinase activators described herein for use in preparation of a medicament for preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

MLR-1023 was evaluated in an animal model of cytokine storm using a 7-day dosing paradigm at 2 dose levels and using dexamethasone as a positive control because it is well-established as an immunosuppressant that significantly depresses key cytokines associated with LPS-induced inflammation such as TNF-∝ and IL-6. It was determined by both forms of measuring pulmonary edema that MLR-1023 demonstrated a significant dose-dependent reduction in pulmonary edema. By one measure (Evans Blue assay) the effect of MLR-1023 was greater than that of dexamethasone and comparable to that of animals that received no LPS.

### Example 1: Effect of MLR-1023 and Dexamethasone in ARDS and Pulmonary Permeability

Intranasal administration of LPS: The LPS challenge was carried out according to Ma et al., Inflammation, 2019, 42, 1901-1912. Briefly, mice were anesthetized by isoflurane and intranasal administration of LPS 50 μg (in 50 μl) was instilled into the nares in one bolus. Mice were then removed to their home cage to recover until fully awake.

EBD administration and EBD assay: The Evan's Blue assay was conducted according to Ma et al. (2019). Briefly, the mice were administered Evan's Blue dye (EBD) (20 mg/kg) through the tail injection. After 30 minutes of blood circulation, mice were sacrificed and approximately 60 mg of lung tissue was weighed. Then, 0.5 ml of formamide was added to the tissue and extracted for 48 hours at 60° C. The supernatant was centrifuged at 12000×g for 10 minutes and the OD of the supernatant was measured at 620 nm on a microplate reader against a standard curve of EBD (4-80 μg/ml).

Wet/Dry ratio assay: The Wet/Dry ratio assay was conducted according to Ma et al. (2019). Briefly, the left lung was extracted and all of the blood stains were wiped off from the lung surface. The wet weight of the lung was recorded. The tissue was dried at 60° C. for 48 hours, the dry weight was recorded, and the W/D ratio was calculated.

Compound dosing: Animals in groups 2-5 were dosed according to the Table 1 for 6 days. On day 7, 1 hour after the last dose, animals were intranasally treated with LPS. Dexamethasone (20 mg/kg PO) was administered 1 hour prior to LPS treatment. Six hours after the instillation of LPS, animals received an IV dose of EBD. Thirty minutes later, animals were sacrificed, and the lungs were collected as previously described.

TABLE 1
Compound dosing

| | | | | | | Takedown, | |
|---|---|---|---|---|---|---|---|
| | | | Days of | Group | | time after | Evaluations/ |
| Group | Treatment | Dose | dosing | size | LPS | LPS | Endpoints |
| 1 | Untreated | NA | NA | 8 | NA | 6 Hours | EBD injected 30 |
| 2 | Vehicle | 0 | QD for | | 0 | | min prior to TD |
| | | IP | 7 days, | | IN | | (5 ml/kg IV) |
| 3 | Vehicle/LPS | 10 ml/kg | Last dose | | 50 ug | | EBD assay (60 |
| | | IP | 1 hr prior | | IN | | mg of right lung |
| 4 | MLR- | 30 mg/kg | to LPS | | | | collected) |
| | 1023/LPS | IP | | | | | W/D assay (left |
| 5 | MLR- | 100 mg/kg | | | | | lung collected) |
| | 1023/LPS | IP | | | | | |
| 6 | Dexamethasone/ | 20 mg/kg | 1 | | | | |
| | LPS | PO | 1 hr prior | | | | |
| | | | to LPS | | | | |

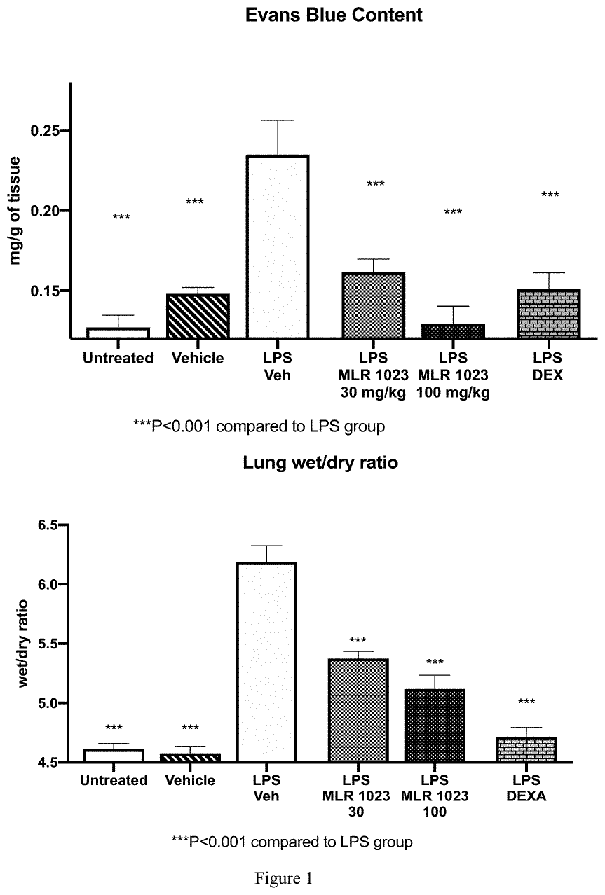

Results: MLR-1023 exhibited a dose-dependent reduction of pulmonary extravasation by two independent measures in a mouse model of cytokine storm. This study was conducted in C57Bl/6J male mice age 6-7 weeks. Animals treated with MLR-1023 were dosed IP, QD for 6 days and then on the 7th day, 1 hour prior to LPS challenge. Dexamethasone was administered PO 1 hour prior to LPS challenge. FIG. 1 shows representative results. ***p<0.001 compared to LPS vehicle control using Fisher's LSD test (n=8).

Treatment of animals with intranasally administered LPS produced a significant increase in wet/dry ratio and Evans Blue dye content in the lung compared to sham and vehicle-treated animals, indicating that this is the result of plasma extravasation. Treatment with MLR-1023 produced a significant dose dependent decrease in Evans Blue content and wet/dry ratio compared to LPS/Vehicle group, indicating that MLR-1023 reduced plasma extravasation. Treatment with dexamethasone produced a significant decrease in Evans Blue content and wet/dry ratio compared to LPS/Vehicle group confirming efficacy of this treatment as it is well-established that dexamethasone significantly reduces the cytokine “storm” associated with LPS administration.

### Example 2: Effect of Acute and Sub-Chronic Treatment with MLR-1023 in ARDS and Pulmonary Permeability

Intranasal administration of LPS: The LPS challenge was carried out according to Ma et al. (2019). Briefly, mice were anesthetized by isoflurane and intranasal administration of LPS 50 μg (in 50 μl) was instilled into the nares in one bolus. Mice were then removed to their home cage to recover until fully awake.

EBD administration and EBD assay: The Evan's Blue assay was conducted according to Ma et al. (2019). Briefly, the mice were administered EBD (20 mg/kg) through the tail injection and after 30 minutes of blood circulation, mice were sacrificed and approximately 60 mg of lung tissue was weighed. Then, 0.5 ml of formamide was added to the tissue and extracted for 48 hours at 60° C. The supernatant was centrifuged at 12000×g for 10 minutes and the OD of the supernatant was measured at 620 nm on a microplate reader against a standard curve of EBD (4-80 μg/ml).

Wet/Dry ratio assay: The Wet/Dry ratio assay was conducted according to Ma et al. (2019). Briefly, the left lung was extracted, all of the blood stains was wiped off from the lung surface, and the wet weight of the lung was recorded. The tissue was dried at 60° C. for 48 hours, the dry weight was recorded, and the W/D ratio was calculated.

Compound dosing: Animals in groups 2-5 were dosed according to the Table 2 for 6 days. Group 3 animals received 6 doses of MLR-1023 QD and a 7th dose 1 hour prior to LPS. Group 4 animals received 6 doses of MLR-1023 QD and Vehicle 1 hour prior to LPS. Group 5 animals received 6 Vehicle doses QD and 1 dose of MLR-1023 1 hour prior to LPS. On day 7, 1 hour after the last dose, animals were intranasally treated with LPS. Six hours after the instillation of LPS, animals received an IV dose of EBD. Thirty minutes later, animals were sacrificed, and the lungs were collected as previously described.

TABLE 2
Compound dosing

| | | MLR- | | | | Takedown, | |
|---|---|---|---|---|---|---|---|
| | | 1023 | Days of | Group | | time after | Evaluations/ |
| Group | Treatment | dose | dosing | size | LPS | LPS | Endpoints |
| 1 | Vehicle | 0 | QD for 7 | 8 | 0 | 6 Hours | Blood/plasma |
| | | IP | days, | | IN | | collection for |
| 2 | Vehicle/LPS | 0 | Last dose | | 50 μg | | IL-6, and |
| | | IP | 1 hr prior | | IN | | TNF alpha |
| | | | to LPS | | | | measurements |
| 3 | MLR- | 100 mg/kg | QD for 7 | | | | Left lung |
| | 1023/LPS | IP | days, | | | | weight for |
| | | | Last dose | | | | W/D ratio |
| | | | 1 hr prior | | | | Right lung for |
| | | | to LPS | | | | EBD assay |
| 4 | | | QD for 7 | | | | |
| | | | days, | | | | |
| | | | 0 dose | | | | |
| | | | (Vehicle) | | | | |
| | | | 1 hr prior | | | | |
| | | | to LPS | | | | |
| 5 | | | QD for 1 | | | | |
| | | | day | | | | |
| | | | 1 hr prior | | | | |
| | | | to LPS | | | | |

IL-6 and TNF-alpha Levels: Cytokine levels were measured by ELISA using commercially available kits according to the manufacturer's recommended methods.

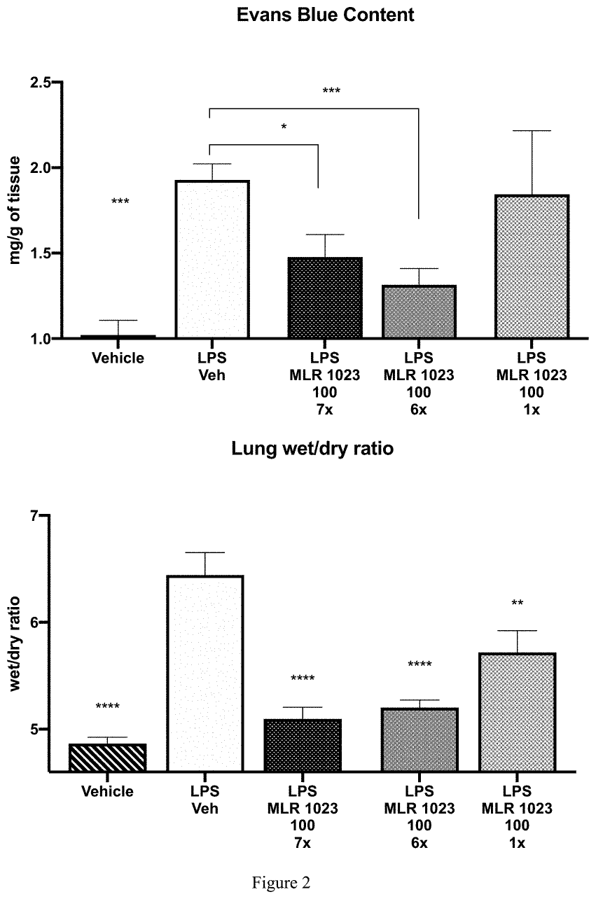

Results: MLR-1023 provided prophylactic benefit against pulmonary edema in a model of cytokine storm which lasts well after drug levels have cleared. This study was conducted in C57B1/ 6J male mice age 6-7 weeks. Animals treated with MLR-1023 were dosed IP, QD for either 6 days with vehicle administration on the 7th day (6×), 1 hour prior to LPS challenge (1×), or, as in the previous study 6 days plus on the 7th day 1 hour prior to LPS challenge (7×). FIG. 2 shows representative results. **p<0.01, ***p<0.001, ****p<0.0001 compared to LPS vehicle control using Fisher's LSD test except in the Evans Blue Content for MLR-1023 7× and 6× were compared to LPS vehicle using paired T-test since the high variability in the 1× group confounded the multiple comparisons analysis with Fisher's LSD test (n=8).

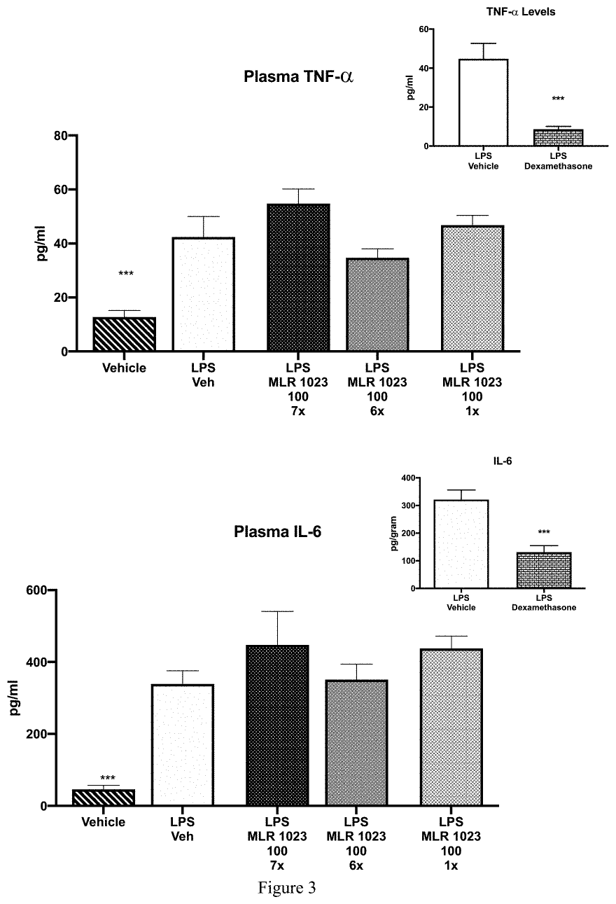

MLR-1023 does not alter cytokine levels in a model of cytokine storm. The same animals as shown in FIG. 2 were evaluated for cytokine levels. Although not included in this study, dexamethasone markedly and reliably reduces TNF-∝ and IL-6 levels. Historical representative data is included for comparison. FIG. 3 shows representative results. ***p<0.001 compared to LPS vehicle control using Fisher's LSD (n=8).

Intranasally administered LPS produced a significant increase in wet/dry ratio and Evans Blue dye content in the lung compared to vehicle-treated animals. Presumably, this is the result of plasma extravasation. Treatment with MLR-1023 (all dosing paradigms) produced a significant decrease in wet/dry ratio compared to LPS/Vehicle group, indicating reduced plasma extravasation, although this effect was more pronounced in animals that had been chronically treated. Treatment with MLR-1023 (6 doses) produced a significant decrease in Evans Blue dye content in the lungs compared to LPS/Vehicle group, indicating reduced plasma extravasation. MLR-1023 treatment in the same 6-dose paradigm plus an acute dose 1 hour before LPS challenge yielded a comparable level of reduced plasma extravasation. Treatment of MLR-1023 for a single dose prior to LPS challenge did not yield reduction in plasma extravasation as judged by Evan's Blue. Intranasally administered LPS produced a significant increase in plasma IL-6 and TNF-α concentrations compared to vehicle-treated animals. Treatment with MLR-1023 (all dosing paradigms) did not produce significant effects in comparison to LPS/vehicle group indicating that the reduction in pulmonary extravasation associated with MLR-1023 treatment was not by way of reduced immune response.

In summary, MLR-1023 was administered daily (QD) by intraperitoneal injection for 6 days and on the 7th day, 1 hour prior to an LPS challenge. A cytokine storm was induced by intranasal administration of LPS into the lungs. Six hours after animals were sacrificed and pulmonary edema was assessed by two methods. One lung was evaluated for a wet weight/dry weight ratio by weighing the lung before and after desiccation. The other lung was evaluated by Evans Blue assay. This was achieved by intravenous injection with Evans Blue dye 30 minutes prior to sacrificing the animals. The lung being assessed in the Evans Blue assay was then processed for Evans Blue extraction and the amount of Evans Blue in the elution fluid was measured by spectrophotometry. If lung barrier is maintained, relatively little Evans Blue leaks from blood vessels into the lungs. If pulmonary vessels are leaky, then relatively more Evans Blue enter the lungs and will be extracted in this procedure.

How a lyn kinase activator should be administered for therapeutic benefit was addressed by separating the 7-day dosing paradigm described previously into two components, a sub-chronic 6-day paradigm where animals were dosed daily for 6 days and then LPS-challenged the following day without further MLR-1023 administration. Another group received MLR-1023 administration 1 hour before LPS-challenge but only vehicle administration for the 6 days prior to that. A third group, for comparison, received the same 7-day paradigm as in the first study with both sub-chronic QD administration and a dose 1 hour prior to LPS. The results showed that most or all of the benefit for MLR-1023 administration was gained in 6 days of sub-chronic administration and relatively little benefit was gained from the administration 1 hour prior to LPS challenge. This was surprising and unexpected in that the plasma half-life of MLR-1023 in mice is about 1.5 hours. Therefore, in animals administered MLR-1023 for 6 days and then not receiving any MLR-1023 for 24 hours before LPS challenge, at which point all MLR-1023 would be cleared from their bodies, all or nearly all of the therapeutic benefit was attained.

To test the hypothesis that MLR-1023 was not mediating its benefit on pulmonary edema by suppressing the immune response, TNF-∝ and IL-6 levels in the plasma from the animals in this study were evaluated. The analysis revealed MLR-1023 administration was not associated with any shift in cytokine levels consistent with the notion that the reduction in pulmonary edema arose from improved integrity of the pulmonary epithelium and not from surpassed immune response.

In conclusion, the findings are consistent with MLR-1023 providing some transformative changed to the pulmonary barrier that remained intact well after the drug had cleared from the body. In a COVID-19 setting, it is inferred that MLR-1023 would best be administered prophylactically in subjects who have COVID-19 before serious pulmonary complications have developed in order to prevent the development of pulmonary edema.

### Example 3: The Effect of Sub-Chronic Treatment with MLR-1023 on Survival Rate of Mice in an Experimental Influenza Model

Mice: 5-6-week-old male C57Bl/6 mice were obtained from JAX. The animals were housed 4 per cage on a 12 hour light/dark cycle in a ventilated cage rack system and fed standard rodent chow and water ad libitum. Animals were assigned randomly to treatment groups with body weight matched for each group for Day 0, and acclimated for at least 3 days prior to futher experiments.

Study Design: Mice (28) were assigned randomly into three groups (8-10 animals per group, see Table 3). Mice were administered a single 50 μl intranasal dose of influenza virus (7.5 Lg EID₅₀ in saline). Daily 10 ml/kg (100 mg/kg) dose of MLR-1023 (in 30% HPBCD solution) was administered intraperitonially over a 10-day period.

TABLE 3
Study Design

| | | Group | Flu | Dose and | Duration | Evaluations/ |
|---|---|---|---|---|---|---|
| Group | Treatment | Size | Dose | Route | (Days) | Endpoints |
| 1 | Vehicle/ | 8 | 0 | Vehicle IP | 10, QD | Daily: |
| | Vehicle | | | | | BW and mortality |
| 2 | Influenza/ | 10 | 7.5 Lg | Vehicle IP | 10, QD | On day 11: |
| | Vehicle | | EID₅₀ | | | Plasma and lung |
| 3 | Influenza/ | 10 | 7.5 Lg | MLR- 1023 | 10, QD | collection in all |
| | MLR- 1023 | | EID₅₀ | 100 mg/kg IP | | surviving animals |

Intranasal administration of influenza virus: Influenza virus A/PR/8/34 H1N1 allantoic fluid was obtained from Charles River Laboratories (Batch #3XP160513). The virus titer in the supplied material was 10^{10.5} EID₅₀ (embryonic infective dose for 50% of chicken embryos). Allantoid fluid was diluted 1000 times with sterile saline to obtain 10^{7.5} EID₅₀ (7.5 Lg EID₅₀) dose.

Mice were anesthetized by isoflurane. For intranasal administration of Influenza virus H1N1 A/PR/8/34, 50 μl of the 7.5 Lg EID₅₀ dose was instilled into the nares in one bolus (25 μl per nare). Mice were then moved to their home cage to recover until fully awake.

Animals were treated with vehicle or MLR-1023 (100 mg/kg) IP for 10 days, beginning on day 1, approximately 4 hours after inoculation with influenza virus. Daily body weights and mortality rate was observed for 10 days.

Results: One purpose of this study was to evaluate whether MLR-1023 exerts its therapeutic effect when administered chronically in the mouse model of experimental influenza infection as determined by the survival rate.

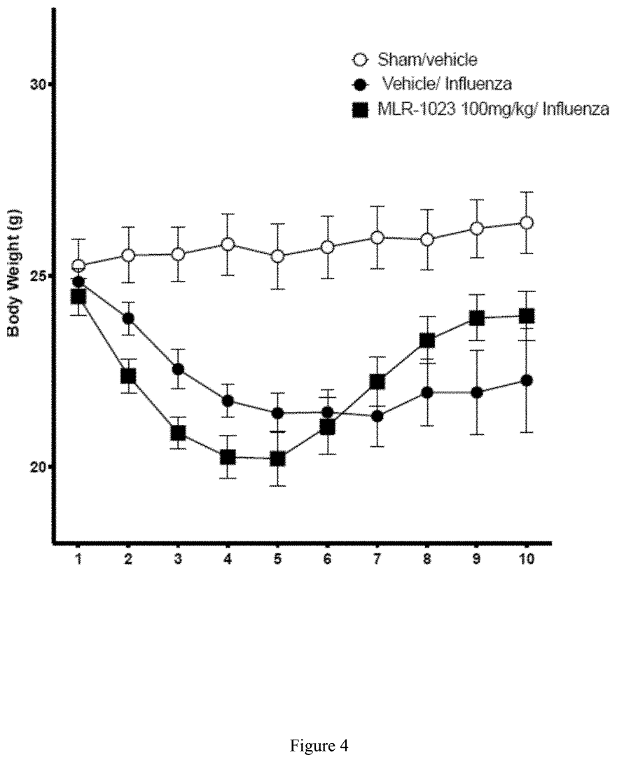

Daily Body Weight: Animals were inoculated with saline or influenza virus at 10^{7.5} EID₅₀ on day 1 and treated with vehicle or MLR-1023 QD. Data are mean±SEM and analyzed by two-way ANOVA with Fisher's uncorrected LSD test. ANOVA revealed a significant effect of treatments (P<0.0001) and time (P<0.0001). There was a significant treatment x time interaction (P=0.0033). There was no significant difference in body weight between Vehicle/influenza group and MLR-1023/Influenza group on days 8, 9 and 10. Inoculation of animals with the influenza virus produced a significant decrease in body weights compared to sham-treated animals. MLR-1023 did not produce a significant effect on the body weight as compared to Influenza/Vehicle treated animals (see, FIG. 4).

Overall survival rate: Animals were inoculated with saline or influenza virus at 10^{7.5} EID₅₀ on day 1 and treated with vehicle or MLR-1023 QD. Data are representing percentage of survival and analyzed by Log-rank Mantel-Cox and Gehan-Breslow-Wilcoxon tests, detecting a significant difference between Influenza/Vehicle and Influenza/MLR-1023 groups (P=0.041).

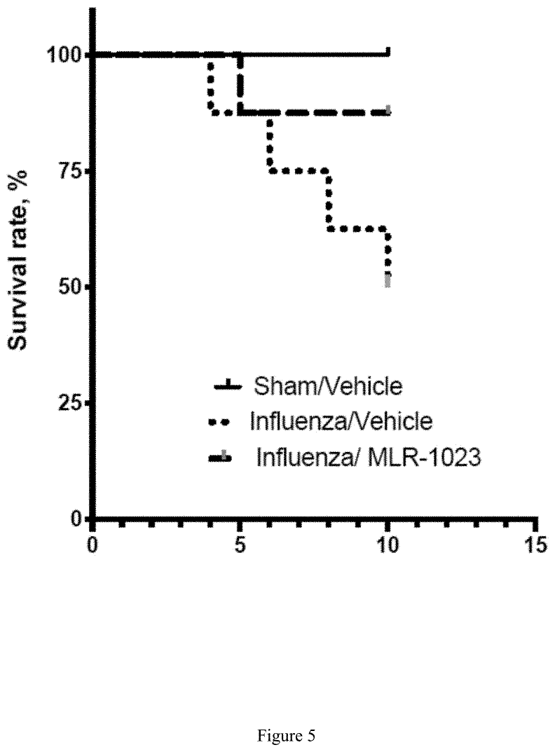

Treatment with MLR-1023 produced a significant benefit on the overall survival rate of animals treated with influenza virus. 87.5% of animals receiving QD treatment with MLR-1023 survived, compared to 50% of animals survived in vehicle-treated group (P<0.05, Gehan-Breslow-Wilcoxon test) (see, FIG. 5).

### Example 4: The Effect of Sub-Chronic Treatment with MLR-1023 on ARDS and Pulmonary Permeability in an Experimental Influenza Model

Mice: 5-6-week-old male C57Bl/6 mice were obtained from JAX. The animals were housed 4 per cage on a 12-hour light/dark cycle in a ventilated cage rack system and fed standard rodent chow and water ad libitum. Animals were assigned randomly to treatment groups with body weight matched for each group for Day 0, and acclimated for at least 3 days prior to futher experiments.

Study Design: Mice (31) were assigned randomly into three groups (7-12 animals per group, see Table 4). Mice were administered a single 50 μl intranasal dose of influenza virus (6.5 Lg EID₅₀ in saline). Daily 10 ml/kg (100 mg/kg) dose of MLR-1023 (in 30% HPBCD solution) was administered intraperitonially over a 7-day period.

TABLE 4
Study Design

| | | Group | Flu | Dose and | Duration | Evaluations/ |
|---|---|---|---|---|---|---|
| Group | Treatment | Size | Dose | Route | (Days) | Endpoints |
| 1 | Vehicle/ | 7 | 0 | Vehicle IP | 7, QD | Wet weight/dry |
| | Vehicle | | | | | weight ratio on Day 7 |
| 2 | Influenza/ | 12 | 6.5 Lg | Vehicle IP | 7, QD | |
| | Vehicle | | EID₅₀ | | | |
| 3 | Influenza/ | 12 | 6.5 Lg | MLR- 1023 | 7, QD | |
| | MLR- 1023 | | EID₅₀ | 100 mg/kg IP | | |

Intranasal administration of influenza virus: Influenza virus A/PR/8/34 H1N1 allantoic fluid was obtained from Charles River Laboratories (Batch #3XP160513). The virus titer in the supplied material was 10^{10.5} EID₅₀ (embryonic infective dose for 50% of chicken embryos). Allantoid fluid was diluted 10,000 times with sterile saline to obtain 10^{6.5} EID₅₀ (6.5 Lg EID₅₀) dose.

Mice were anesthetized by isoflurane. For intranasal administration of Influenza virus H1N1 A/PR/8/34, 50 μl of the 6.5 LgEID₅₀ dose was instilled into the nares in one bolus (25 μl per nare). Mice were then moved to their home cage to recover until fully awake.

Animals were treated with vehicle or MLR-1023 (100 mg/kg) IP for 7 days, beginning on day 1, approximately 4 hours after inoculation with influenza virus.

Wet/Dry ratio assay: On Day 7 after inoculation with virus and MLR-1023 treatment, animals were sacrificed, lungs collected, and the Wet/Dry ratio assay was conducted according to Ma et al. (2019). Briefly, the left lung was extracted and all of the blood stains were wiped off from the lung surface. The wet weight of the lung was recorded. The tissue was dried at 60° C. for 48 hours, the dry weight was recorded, and the W/D ratio was calculated.

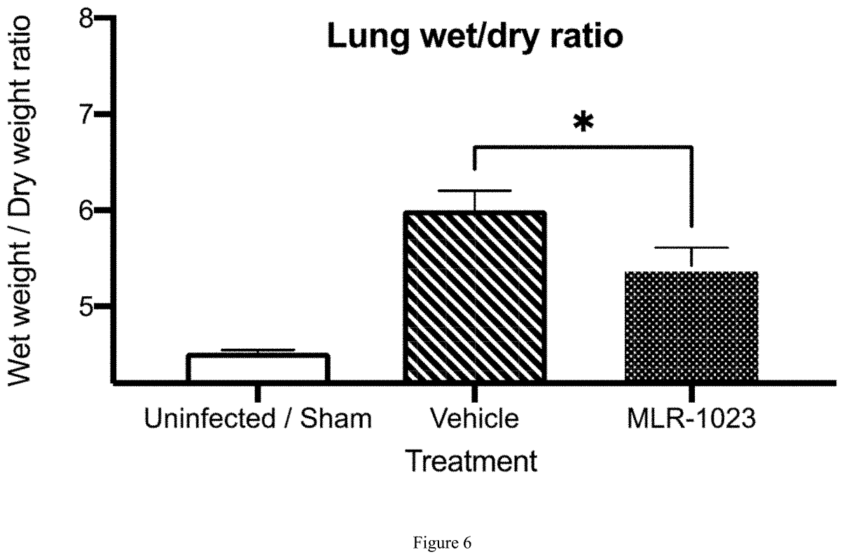

Results: MLR-1023 significantly reduced wet weight/dry weight ratio and thereby reduced pulmonary edema in influenza infected mice. FIG. 6 shows representative results (*p<0.05, inflenza-infected/MLR-1023-treated compared to influenza-infected/vehicle-treated control using Fisher's LSD test).

### Example 5: The Effect of Sub-Chronic Treatment with MLR-1023 on Immunoglobulin Levels in an Experimental Influenza Model

Mice: 5-6-week-old male C57Bl/6 mice were obtained from JAX. The animals were housed 4 per cage on a 12-hour light/dark cycle in a ventilated cage rack system and fed standard rodent chow and water ad libitum. Animals were assigned randomly to treatment groups with body weight matched for each group for Day 0, and acclimated for at least 3 days prior to futher experiments.

Study Design: Mice (31) were assigned randomly into three groups (7-12 animals per group, see Table 5). Mice were administered a single 50 μl intranasal dose of influenza virus (6.5 Lg EID₅₀ in saline). Daily 10 ml/kg (100 mg/kg) dose of MLR-1023 (in 30% HPBCD solution) was administered intraperitonially over a 7-day period.

TABLE 5
Study Design

| | | Group | Flu | Dose and | Duration | Evaluations/ |
|---|---|---|---|---|---|---|
| Group | Treatment | Size | Dose | Route | (Days) | Endpoints |
| 1 | Vehicle/ | 7 | 0 | Vehicle IP | 12, QD | Day 12 Serum levels of: |
| | Vehicle | | | | | Total IgG |
| 2 | Influenza/ | 12 | 6.5 Lg | Vehicle IP | 12, QD | Total IgM |
| | Vehicle | | EID₅₀ | | | Influenza-sepcific Ab |
| 3 | Influenza/ | 12 | 6.5 Lg | MLR- 1023 | 12, QD | |
| | MLR- 1023 | | EID₅₀ | 100 mg/kg IP | | |

Intranasal administration of influenza virus: Influenza virus A/PR/8/34 H1N1 allantoic fluid was obtained from Charles River Laboratories (Batch #3XP160513). The virus titer in the supplied material was 10^{10.5} EID₅₀ (embryonic infective dose for 50% of chicken embryos). Allantoid fluid was diluted 10,000 times with sterile saline to obtain 10^{6.5} EID₅₀ (6.5 Lg EID₅₀) dose.

Mice were anesthetized by isoflurane. For intranasal administration of Influenza virus H1N1 A/PR/8/34, 50 μl of the 6.5 LgEID₅₀ dose was instilled into the nares in one bolus (25 μl per nare). Mice were then moved to their home cage to recover until fully awake.

Animals were treated with vehicle or MLR-1023 (100 mg/kg) IP for 12 days, beginning on day 1, approximately 4 hours after inoculation with influenza virus.

Immunoglobulin Levels: On Day 12 after inoculation with virus and MLR-1023 treatment, animals were sacrificed, blood collected by cardiac puncture, serum prepared and total IgG, total IgM and Influenza-specific antibody measured by ELISA using commercially available kits according to the manufacturer.

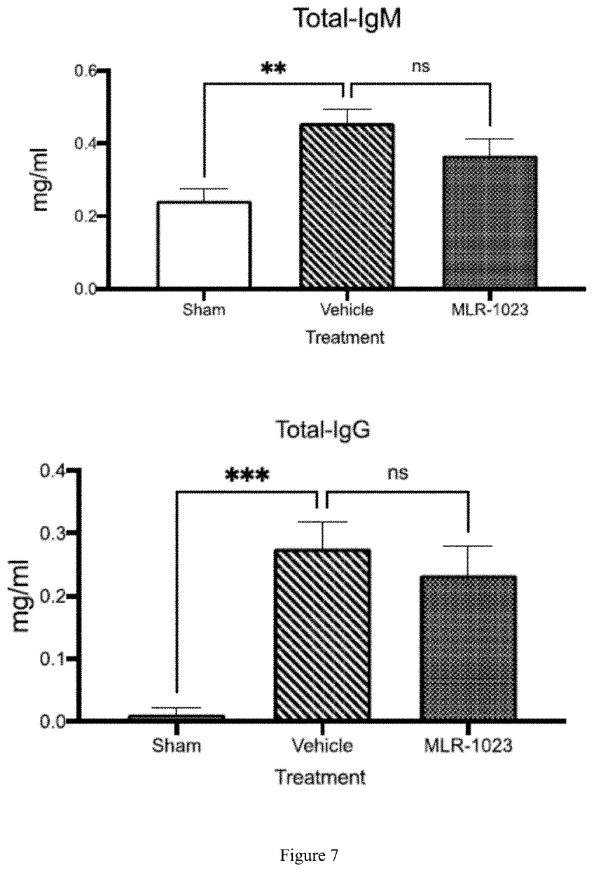

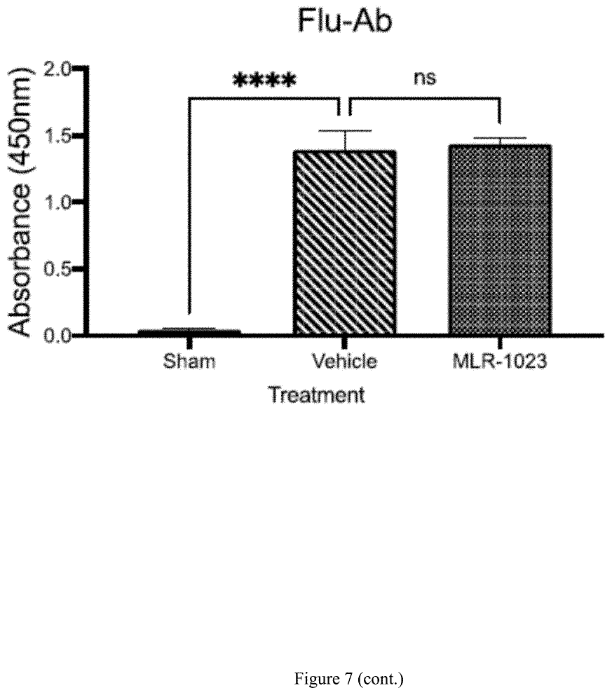

Results: Influenza infection significantly increased levels of IgG, IgM and influenza-specific antibody compared to uninfected controls. MLR-1023 did not alter any immunoglobulin level (either total IgG, total IgM or influenza-specific antibody) in influenza infected mice on Day 12 after infection when MLR-1023 was administed daily over the course of the infection, compared to influenza-infected/vehicle treated controls. FIG. 7 shows representative results (**p<0.01, ***p<0.001, ****p<0.0001, for influenza-infected/vehicle-treated compared to non-infected vehicle-treated control using Fisher's LSD test). These results are consistent with the notion that MLR-1023 reduces pulmonary edema (leakiness) by direct action on strengthening the pulmonary barrier and not by modulating immune function, consistent with the findings presented in Example 2.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of preventing, delaying, or reducing the severity of pulmonary extravasation, pulmonary edema, pneumonia, fluid in the lung, acute respiratory distress syndrome, plasma extravasation, and/or pulmonary conditions that develop as a result of hypercytokinemia (cytokine storm syndrome) in a mammal having a medical condition, the method comprising administering to the mammal a compound having the formula: 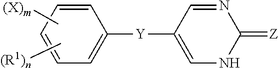
wherein:
R¹ is an alkyl group;
X is a halogen;
Y is O, S, or NH;
Z is O or S;
n is an integer from 0 to 5 and m is 0 or 1, wherein m+n is less than or equal to 5; or a pharmaceutically acceptable salt thereof.

2. The method of claim 1, wherein the alkyl group is methyl and n is 1.

3. The method of claim 1, wherein the halogen is chlorine and m is 1.

4. The method of claim 1, wherein Y is O.

5. The method of claim 1, wherein Z is O.

6. The method of claim 1, wherein R¹ is methyl, Y is O, Z is O, n is 1, and m is 0.

7. The method of claim 6, wherein R¹ is in the meta position.

8. The method of claim 1, wherein X is chlorine, Y is O, Z is O, n is 0, and m is 1.

9. The method of claim 8, wherein X is in the meta position.

10. The method of claim 1, wherein the lyn kinase activator is of the formula: 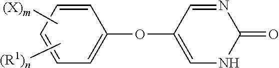
wherein:
R¹ is an alkyl group;
X is a halogen; and
n is an integer from 0 to 5 and m is 0 or 1, wherein m+n is less than or equal to 5; or a pharmaceutically acceptable salt thereof.

11. The method of claim 10, wherein the alkyl group is methyl and n is 1.

12. The method of claim 10, wherein the halogen is chlorine and m is 1.

13. The method of claim 10, wherein R¹ is methyl, n is 1, and m is 0.

14. The method of claim 13, wherein R¹ is in the meta position.

15. The method of claim 12, wherein X is chlorine, n is 0, and m is 1.

16. The method of claim 15, wherein X is in the meta position.

17. The method of claim 1, wherein the lyn kinase activator is of the formula: 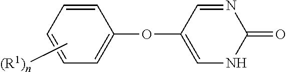
wherein R¹ is an alkyl group and n is an integer from 0 to 5; or a pharmaceutically acceptable salt thereof.

18. The method of claim 17, wherein R¹ is methyl, n is 1.

19. The method of claim 18, wherein R¹ is in the meta position.

20. The method of claim 1, wherein the lyn kinase activator is of the formula: 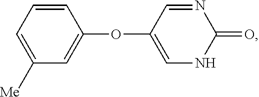 [SMILES: Cc1cccc(Oc2cnc(=O)[nH]c2)c1]
or a pharmaceutically acceptable salt thereof.

21. The method of claim 1, wherein the lyn kinase activator is of the formula: 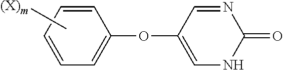
wherein X is a halogen and m is an integer from 0 to 1; or a pharmaceutically acceptable salt thereof.

22. The method of claim 21, wherein X is chloro and m is 1.

23. The method of claim 22, wherein X is in the meta position.

24. The method of claim 1, wherein the lyn kinase activator is of the formula: 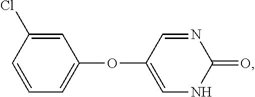 [SMILES: O=c1ncc(Oc2cccc(Cl)c2)c[nH]1]
or a pharmaceutically acceptable salt thereof.

25. The method of claim 1, wherein the lyn kinase activator is of the formula: 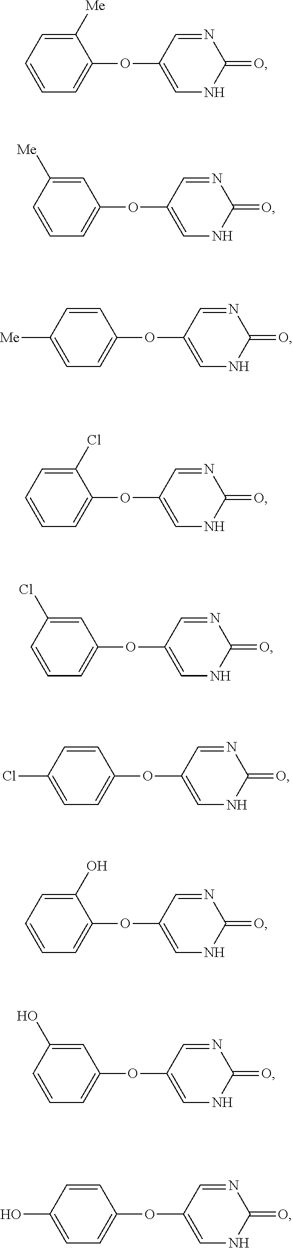 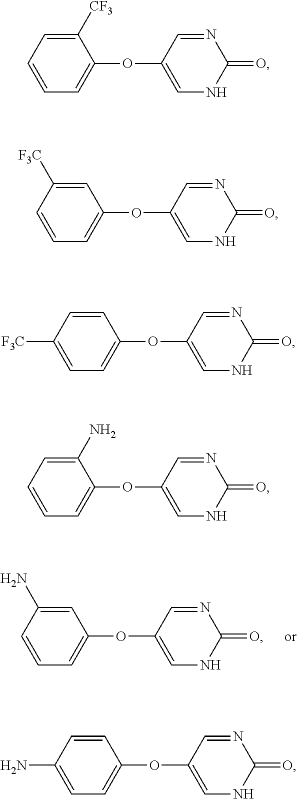 [SMILES: O=c1ncc(Oc2ccccc2C(F)(F)F)c[nH]1 ; O=c1ncc(Oc2cccc(C(F)(F)F)c2)c[nH]1 ; O=c1ncc(Oc2ccc(C(F)(F)F)cc2)c[nH]1 ; Nc1ccccc1Oc1cnc(=O)[nH]c1 ; Nc1cccc(Oc2cnc(=O)[nH]c2)c1 ; Nc1ccc(Oc2cnc(=O)[nH]c2)cc1]
or a pharmaceutically acceptable salt thereof.

26. The method of claim 1, wherein the medical condition is a viral infection, a bacterial infection, a cardiovascular condition, inhalation of a harmful agent, or a head or chest injury.

27. The method of claim 26, wherein the viral infection is an influenza virus infection, a corona virus infection, a human rhinovirus (HRV) infection, a respiratory syncytial virus (RSV) infection, a parainfluenza virus (PIV) infection, a human metapneumovirus (hMPV) infection, or an adenovirus infection.

28. The method of claim 27, wherein the influenza virus infection is an influenza A H1N1 virus infection, and the corona virus infection is a severe acute respiratory syndrome coronavirus 2 (SARS-CoV-2) infection, a severe acute respiratory syndrome coronavirus 1 (SARS-CoV-1) infection, or a Middle East respiratory syndrome virus (MERS) infection.

29. The method of claim 26, wherein the bacterial infection is bacterial pneumonia or sepsis.

30. The method of claim 26, wherein the cardiovascular condition is acute heart failure.

31. The method of claim 26, wherein the harmful agent is smoke or a noxious chemical fume.

32. The method of claim 1, wherein the compound is administered to the mammal every 1 to 3 hours, every 4 to 6 hours, every 7 to 9 hours, every 10 to 12 hours, every 13 to 15 hours, every 16 to 18 hours, every 19 to 21 hours, or every 22 to 24 hours after the mammal has been diagnosed as having the medical condition.

33. The method of claim 1, wherein the amount of the compound administered to the mammal is from about 50 μg to about 1,000 mg, from about 100 μg to about 500 mg, from about 250 μg to about 100 mg, from about 500 μg to about 50 mg, from about 1 mg to about 40 mg, from about 5 mg to about 25 mg, or from about 10 mg to about 20 mg.

34. The method of claim 1, further comprising administering to the mammal any one or more of a statin, a PPAR agonist, a bile-acid-binding resin, niacin, nicotinic acid, a RXR agonist, an anti-obesity drug, a hormone, a tyrophostine, a sulfonylurea-based drug, a biguanide, an α-glucosidase inhibitor, an apo A-I agonist, a cardiovascular drug, a chemotherapeutic agent, an FXR agonist, a PPARα agonist, a GLP-1 agonist, a PPARα/δ dual agonist, an ACC inhibitor, a growth factor, a CCR2/5 blocker, an anti-liver disease therapeutic agent, and an anti-inflammatory agent.
